(19) United States
(12) Patent Application Publication
(10) Pub. No.: US 2024/0058424 A1
(43) Pub. Date: Feb. 22, 2024

(54) NBP-14 FOR TREATING ALZHEIMER'S ASSOCIATED WITH DOWN'S SYNDROME

(71) Applicant: NEURO-BIO LTD, Abingdon, Oxfordshire (GB)

(72) Inventor: Susan GREENFIELD, Abingdon (GB)

(21) Appl. No.: 18/044,373

(22) PCT Filed: Sep. 8, 2021
(86) PCT No.: PCT/GB2021/052312

§ 371 Date: Mar. 7, 2023

(30) Foreign Application Priority Data
Sep. 8, 2020 (GB) 2014080.2

(51) Int. Cl. A61K 38/46; A61P 25/28

(52) U.S. Cl. CPC A61K 38/465; A61P 25/28; C12Y 301/01007

(57) ABSTRACT

The invention provides a cyclic polypeptide, derivative or analogue thereof comprising an amino acid sequence derived from the C-terminus of acetylcholinesterase (AChE), or a truncation thereof, for use in treating, preventing or ameliorating Down's syndrome.

The invention relates to Down's syndrome, and in particular to novel pharmaceutical compositions, therapies and methods for treating, preventing or ameliorating Down's syndrome.

Down's syndrome, also known as trisomy 21, is a genetic condition caused by the presence of all, or part of, a third copy of chromosome 21. It is usually associated with physical growth delays, mild to moderate intellectual disability, and characteristic facial features. The average life expectancy for a person with Down's syndrome is between about 50 and 60, and there is no cure or effective treatment for Down's syndrome, though education and proper care have been shown to improve the quality of life to some degree.

Many, but not all, people with Down's syndrome develop dementia when they get older, and this can be associated with, or caused by, Alzheimer's Disease (AD). Indeed, current estimates suggest that 50% or more of people with Down's syndrome will develop dementia due to Alzheimer's disease as they age, and people with Down's usually begin to show symptoms of Alzheimer's in their 50s or 60s. Adults with Down's syndrome develop β-amyloid plaques that are indistinguishable from the plaques found in Alzheimer's disease patients and are strongly associated with a high risk of dementia and cognitive decline (Annus et al 2016, Alzheimer's and Dementia, 538-545). The gene encoding amyloid is located on chromosome 21.

There is, therefore, a need to provide a novel therapy for treating Down's syndrome, and especially a medicament for delaying or preventing early onset dementia and/or cognitive decline in Down's syndrome subjects.

In view of the above, the inventors believe that any compound that is able to reduce or inhibit β-amyloid plaque formation in a person with Down's syndrome would provide a means of preventing early onset dementia and cognitive decline. The inventors therefore investigated the effects of a cyclic peptide derived from the C-terminus of acetylcholinesterase (known as “NBP-14”) on β-amyloid plaque formation and have surprisingly demonstrated that they are able to reduce in vivo β-amyloid plaque formation in mice. In addition, the cyclic peptide, NBP-14, is surprisingly able to reverse in vivo cognitive decline in a transgenic mouse model of AD. Accordingly, the inventors believe that these cyclic peptides may be utilized as a therapeutic agent to treat, prevent or ameliorate Down's syndrome by reducing β-amyloid plaque formation.

Thus, in a first aspect of the invention, there is provided a cyclic polypeptide, derivative or analogue thereof comprising an amino acid sequence derived from the C-terminus of acetylcholinesterase (AChE), or a truncation thereof, for use in treating, preventing or ameliorating Down's syndrome.

In a second aspect, there is provided a method of treating, ameliorating or preventing Down's syndrome, the method comprising, administering, or having administered, to a subject in need of such treatment, a therapeutically effective amount of a cyclic polypeptide, derivative or analogue thereof comprising an amino acid sequence derived from the C-terminus of acetylcholinesterase (AChE), or a truncation thereof.

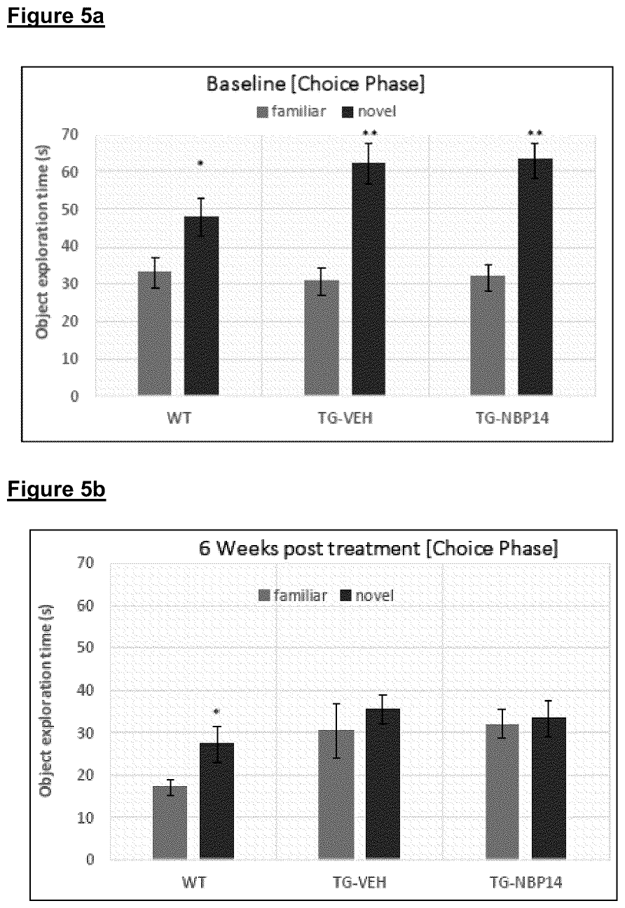

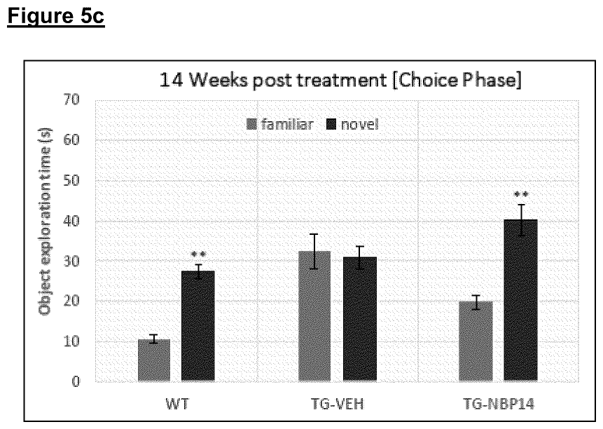

Advantageously, as described in the examples, the inventors intranasally applied a cyclic peptide derived from the C-terminus of acetylcholinesterase (known as “NBP-14”) to transgenic Tg-5XFAD mice, which overexpress mutant human amyloid beta (A4) precursor protein 695 (APP), and which therefore pre-disposes the mice to develop amyloid plaques. Surprisingly, the inventors observed a significant decrease in the intensity of intracellular β-amyloid in the hippocampus and cortex of these Tg-5XFAD mice treated with NBP-14 twice weekly for 6 weeks, compared to vehicle, whilst at 14 weeks, the amyloid had accumulated outside of the cells to form plaques that were significantly reduced by NBP-14 in the cortex, hippocampus and basal forebrain compared to the vehicle-treated cohort. In addition, as shown in FIG. 5c, the inventors were surprised to observe that NBP-14 has a significant protective effect on cognitive decline in transgenic Tg-5XFAD mice otherwise predisposed to develop dementia. Furthermore, surprisingly, NBP-14 was also able to reverse the cognitive decline that was observed in the transgenic mice to a level of performance that was comparable to a wild-type group. The inventors' work has therefore shown that the cyclic peptide derived from the C-terminus of acetylcholinesterase reduces β-amyloid formation and protects from, and reverses, cognitive decline, thereby indicating that cyclic peptides derived from the C-terminus of acetylcholinesterase may be used in the treatment Down's syndrome.

The skilled person would understand that Down's syndrome can also be referred to as trisomy 21.

Preferably, the cyclic polypeptide, derivative or analogue thereof is capable of reducing and/or inhibiting β-amyloid plaque formation in a Down's syndrome person. Plaque formation may preferably be inhibited in the person's hippocampus and/or cortex.

Preferably, the cyclic polypeptide, derivative or analogue thereof is capable of reducing and/or inhibiting phosphorylated Tau (pTau) formation in a Down's syndrome person. Phosphorylated Tau formation is preferably inhibited in the person's hippocampus and/or cortex.

Preferably, the cyclic polypeptide, derivative or analogue thereof is capable of reducing, inhibiting and/or reversing cognitive decline in a Down's syndrome person.

Preferably, the cyclic polypeptide, derivative or analogue thereof is capable of reducing, inhibiting and/or reversing dementia in a Down's syndrome person, more preferably early onset dementia in a Down's syndrome person.

Preferably, the cyclic polypeptide, derivative or analogue thereof is capable of reducing, inhibiting and/or reversing cognitive decline or dementia in a Down's syndrome person who is in their 20's, 30's, 40's, 50's, 60's or 70's. Advantageously, and preferably, the condition is prevented before symptoms ever appear, or before they suffer from a higher rate of cognitive decline, or before dementia sets in.

Cyclic polypeptides are peptide chains whose N- and C-termini are themselves linked together with a peptide bond that forms a circular chain of amino acids.

The term “derivative or analogue thereof” can mean a polypeptide within which amino acid residues are replaced by residues (whether natural amino acids, non-natural amino acids or amino acid mimics) with similar side chains or peptide backbone properties.

Additionally, the terminals of such peptides may be protected by N- and/or C-terminal protecting groups with similar properties to acetyl or amide groups.

Derivatives and analogues of peptides according to the invention may also include those that increase the peptide's half-life in vivo. For example, a derivative or analogue of the peptides of the invention may include peptoid and retropeptoid derivatives of the peptides, peptide-peptoid hybrids and D-amino acid derivatives of the peptides.

Peptoids, or poly-N-substituted glycines, are a class of peptidomimetics whose side chains are appended to the nitrogen atom of the peptide backbone, rather than to the alpha-carbon, as they are in amino acids. Peptoid derivatives of the peptides of the invention may be readily designed from knowledge of the structure of the peptide. Retropeptoids (in which all amino acids are replaced by peptoid residues in reversed order) are also suitable derivatives in accordance with the invention. A retropeptoid is expected to bind in the opposite direction in the ligand-binding groove, as compared to a peptide or peptoid-peptide hybrid containing one peptoid residue. As a result, the side chains of the peptoid residues are able point in the same direction as the side chains in the original peptide.

The term “derived from” can mean an amino acid sequence, which is a derivative or a modification of an amino acid sequence that is present in, or forms, the C-terminus of AChE, and portion thereof.

The term “truncation thereof” can mean the cyclic polypeptide derived from AChE is reduced in size by the removal of amino acids. The reduction of amino acids may be achieved by removal of residues from the C- or N-terminal of the peptide prior to cyclisation into the cyclic polypeptide of the invention, or may be achieved by deletion of one or more amino acids from within the core of the peptide prior to cyclisation.

Acetylcholinesterase is a serine protease that hydrolyses acetylcholine, and will be well-known to the skilled person. The major form of acetylcholinesterase, which is found in the brain, is known as tailed acetylcholinesterase (T-AChE). The protein sequence of one embodiment of human tailed acetylcholinesterase (Gen Bank: AAA68151.1) is 614 amino acids in length, and is provided herein as SEQ ID No:1, as follows:

```
[SEQ ID No: 1]
    1 mrppqcllht pslaspllll llwllgggvg
        aegredaell vtvrggrlrg irlktpggpv
  61 saflgipfae ppmgprrflp pepkqpwsgv
        vdattfqsvc yqyvdtlypg fegtemwnpn
121 relsedclyl nvwtpyprpt sptpvlvwiy
        gggfysgass ldvydgrflv qaertvlvsm
181 nyrvgafgfl alpgsreapg nvglldqrla
        lqwvqenvaa fggdptsvtl fgesagaasv
241 gmhllsppsr glfhravlqs gapngpwatv
        gmgearrrat qlahlvgcpp ggtggndtel
301 vaclrtrpaq vlvnhewhvl pqesvfrfsf
        vpvvdgdfls dtpealinag dfhglqvlvg
361 vvkdegsyfl vygapgfskd neslisraef
        lagvrvgvpq vsdlaaeavv lhytdwlhpe
421 dparlreals dvvgdhnvvc pvaqlagrla
        aqgarvyayv fehrastlsw plwmgvphgy
481 eiefifgipl dpsrnytaee kifaqrlmry
        wanfartgdp neprdpkapq wppytagaqq
541 yvsldlrple vrrglraqac afwnrflpkl
        lsatdtldea erqwkaefhr wssymvhwkn
601 qfdhyskqdr csdl
```

It will be appreciated that the first 31 amino acid residues of SEQ ID No:1 are removed while the protein is released, thereby leaving a 583 amino acid sequence. Accordingly, it is preferred that the cyclic polypeptide, derivative or analogue thereof comprises or consists of an amino acid sequence derived from the C-terminus of acetylcholinesterase, or a truncation thereof, wherein the acetylcholinesterase comprises an amino acid sequence substantially as set out in SEQ ID No:1, preferably excluding the 31 amino acids at the N-terminal.

Preferably, the cyclic polypeptide, derivative or analogue thereof comprises or consists of an amino acid sequence derived from the last 300, 200, 100 or 50 amino acids forming the C-terminus of acetylcholinesterase, or a truncation thereof, most preferably wherein the acetylcholinesterase comprises or consists of an amino acid sequence substantially as set out in SEQ ID No:1. The cyclic polypeptide, derivative or analogue thereof preferably comprises or consists of an amino acid sequence derived from the last 40 amino acids forming the C-terminus of acetylcholinesterase, or a truncation thereof The cyclic polypeptide, derivative or analogue thereof preferably comprises or consists of an amino acid sequence derived from the last 30 amino acids forming the C-terminus of acetylcholinesterase, or a truncation thereof.

The cyclic polypeptide, derivative or analogue thereof may comprise or consist of between 4 and 50 amino acids, preferably between 8 and 40 amino acid residues, preferably between 10 and 30 amino acids, more preferably between 9 and 20 amino acids, and most preferably between 10 and 16 amino acids. More preferably, the cyclic polypeptide, derivative or analogue thereof may comprise or consist of between 13 and amino acid residues.

Preferably, the cyclic polypeptide, derivative or analogue thereof, comprises between 4 and 50 amino acid residues, between 4 and 40 amino acid residues, between 4 and 35 amino acid residues, between 4 and 32 amino acid residues, between 4 and 30 amino acid residues, between 4 and 25 amino acid residues, between 4 and 20 amino acid residues, or between 4 and 15 amino acid residues.

Preferably, the cyclic polypeptide, derivative or analogue thereof, comprises between 5 and 50 amino acid residues, between 5 and 40 amino acid residues, between 5 and 35 amino acid residues, between 5 and 32 amino acid residues, between 5 and 30 amino acid residues, between 5 and 25 amino acid residues, between 5 and 20 amino acid residues, or between 5 and 15 amino acid residues.

Preferably, the cyclic polypeptide, derivative or analogue thereof, comprises between 6 and 50 amino acid residues, between 6 and 40 amino acid residues, between 6 and 35 amino acid residues, between 6 and 32 amino acid residues, between 6 and 30 amino acid residues, between 6 and 25 amino acid residues, between 6 and 20 amino acid residues, or between 6 and 15 amino acid residues.

Preferably, the cyclic polypeptide, derivative or analogue thereof, comprises between 7 and 50 amino acid residues, between 7 and 40 amino acid residues, between 7 and 35 amino acid residues, between 7 and 32 amino acid residues, between 7 and 30 amino acid residues, between 7 and 25 amino acid residues, between 7 and 20 amino acid residues, or between 7 and 15 amino acid residues.

The cyclic polypeptide, derivative or analogue thereof may comprise or consist of between 8 and 40 amino acid residues, preferably between 10 and 30 amino acids, more preferably between 9 and 20 amino acids, and most preferably between 10 and 16 amino acids. More preferably, the cyclic polypeptide, derivative or analogue thereof may comprise or consist of between 13 and 15 amino acid residues.

Preferably, the cyclic polypeptide, derivative or analogue thereof, comprises between 8 and 50 amino acid residues, between 8 and 40 amino acid residues, between 8 and 35 amino acid residues, between 8 and 30 amino acid residues, between 8 and 30 amino acid residues, between 8 and 25 amino acid residues, between 8 and 20 amino acid residues, or between 8 and 15 amino acid residues.

Preferably, the cyclic polypeptide, derivative or analogue thereof, comprises between 9 and 50 amino acid residues, between 9 and 40 amino acid residues, between 9 and 35 amino acid residues, between 9 and 30 amino acid residues, between 9 and 25 amino acid residues, between 9 and 20 amino acid residues, or between 9 and 15 amino acid residues.

Preferably, the cyclic polypeptide, derivative or analogue thereof, comprises between 10 and 50 amino acid residues, between 10 and 40 amino acid residues, between 10 and 35 amino acid residues, between 10 and 30 amino acid residues, between 10 and 25 amino acid residues, between 10 and 20 amino acid residues, or between 10 and 15 amino acid residues.

Preferably, the cyclic polypeptide, derivative or analogue thereof, comprises between 11 and 50 amino acid residues, between 11 and 40 amino acid residues, between 11 and 35 amino acid residues, between 11 and 30 amino acid residues, between 11 and 25 amino acid residues, between 11 and 20 amino acid residues, or between 11 and 15 amino acid residues.

Preferably, the cyclic polypeptide, derivative or analogue thereof, comprises between 12 and 50 amino acid residues, between 12 and 40 amino acid residues, between 12 and 35 amino acid residues, between 12 and 30 amino acid residues, between 12 and 25 amino acid residues, between 12 and 20 amino acid residues, or between 12 and 15 amino acid residues.

Preferably, the cyclic polypeptide, derivative or analogue thereof, comprises between 13 and 50 amino acid residues, between 13 and 40 amino acid residues, between 13 and 35 amino acid residues, between 13 and 30 amino acid residues, between 13 and 25 amino acid residues, between 13 and 20 amino acid residues, or between 13 and 15 amino acid residues.

Preferably, the cyclic polypeptide, derivative or analogue thereof, comprises between 14 and 50 amino acid residues, between 14 and 40 amino acid residues, between 14 and 35 amino acid residues, between 14 and 30 amino acid residues, between 14 and 25 amino acid residues, between 14 and 20 amino acid residues, or between 14 and 15 amino acid residues.

The inventors have prepared three peptide sequences that are derived from the C-terminus of the enzyme acetylcholinesterase (AChE), and which are referred to herein as T30, T14 and T15, where the number corresponds to the amino acid number. AChE is expressed at different stages of development in various forms, all of which have identical enzymatic activity, but which have different molecular compositions. The ‘tailed’ (T-AChE—SEQ ID No: 1) is expressed at synapses and the inventors have previously identified two peptides that could be cleaved from the C-terminus of T-AChE. One of these peptides is a 14 amino acid long peptide referred to as “T14” (SEQ ID No: 3), within the other peptide which is a 30 amino acid long peptide known as “T30” (SEQ ID No: 2). The AChE C-terminal peptide “T14”' has been identified as being the salient part of the AChE molecule responsible for its range of non-hydrolytic actions.

The synthetic analogue (i.e. “T14”), and subsequently the larger and more stable amino acid sequence in which it is embedded (i.e. “T30”) display actions comparable to those reported for ‘non-cholinergic’ AChE, whereas the inert 15 amino acid long peptide within the T30 sequence (i.e. “T15” - SEQ ID No: 4) is without effect (Bond et al 2009 PLoS one Vol: 4 Issue: 3 e4846).

The amino acid sequence of T30 (which corresponds to the last 30 amino acid residues of SEQ ID No:1) is provided herein as SEQ ID No:2, as follows:

[SEQ ID No: 2]
KAEFHRWSSYMVHWKNQFDHYSKQDRCSDL

The amino acid sequence of T14 (which corresponds to the 14 amino acid residues located towards the end of SEQ ID No:1, and lacks the final 15 amino acids found in T30) is provided herein as SEQ ID No:3, as follows:

[SEQ ID No: 3]
AEFHRWSSYMVHWK

The amino acid sequence of T15 (which corresponds to the last 15 amino acid residues of SEQ ID No:1) is provided herein as SEQ ID No:4, as follows:

[SEQ ID No: 4]
NQFDHYSKQDRCSDL

It will be appreciated that any of the sequences represented as SEQ ID No:2-4 can be readily cyclised (or cyclated) to form a cyclic polypeptide, derivative or analogue used in accordance with the first aspect. For example, cyclization of peptides can be achieved by side-chain-to-side-chain, side-chain-to-backbone, or head-to-tail (C-terminus to N-terminus) cyclization techniques. In one preferred embodiment, head-to-tail cyclization is the preferred method by which the cyclic polypeptides are produced. The cyclic polypeptides may be synthesised using either classical solution-phase linear peptide cyclization or resin-based cyclization. Preferred methods for cyclization are described in the Examples. In another preferred embodiment, the polypeptide is produced using a cyclization cleavage approach, in which the cyclic polypeptide is synthesized by cyclization after step-wise linear peptide synthesis. An advantage of this method is that the side-chain does not need to be anchored, making the approach more general. Preferably, prior to use, resultant samples of cyclic peptides can be analysed by MALDI-TOF MS.

Accordingly, a preferred polypeptide, derivative or analogue thereof according to the invention comprises or consists of cyclic SEQ ID No: 2, 3 or 4, or a functional variant or fragment thereof.

The inventors found that cyclized SEQ ID No: 3 (i.e. referred to herein as “cyclized T14”, “CT14” or “NBP-14”) surprisingly reduces β-amyloid plaque formation in the brain.

Accordingly, a most preferred cyclic polypeptide, derivative or analogue thereof used in the invention described herein comprises or consists of cyclic SEQ ID No: 3, or a functional variant or fragment thereof.

It will be appreciated that the cyclic polypeptide, derivative or analogue thereof according to the invention may be used in a medicament, which may be used as a monotherapy (i.e. use of the cyclic polypeptide, derivative or analogue thereof alone), for treating, ameliorating, or preventing Down's syndrome, preferably reducing, inhibiting and/or reversing cognitive decline and/or dementia in a Down's syndrome person. Alternatively, the cyclic polypeptide, derivative or analogue thereof according to the invention may be used as an adjunct to, or in combination with, known therapies for treating, ameliorating, or preventing Down's syndrome.

The cyclic polypeptide according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given, and preferably enables delivery of the cyclic polypeptide across the blood-brain barrier.

It will be appreciated that the efficiency of any treatment for brain disorders, such as cognitive decline or dementia etc., depends on the ability of the candidate therapeutic compound to cross the blood-brain barrier (BBB). The inventor believes that peptides of the size of cyclic T14 (NBP-14) may not gain ready access following oral administration.

Two main strategies may be applied to cross the BBB with a large molecule, such as Cylic T14 (i.e. NBP-14), including: (1) use of nanoparticles as transporters to specifically target the brain and deliver the active compound. This method has successfully been used to deliver peptides, proteins and anticancer drugs deliver to the brain; (2) use of cargo peptides. The addition of such a peptide specifically transported across the BBB allows the transfer of the cyclic peptide through a facilitated manner.

Medicaments comprising cyclic polypeptides according to the invention may be used in a number of ways. For instance, oral administration may be required, in which case the cyclic polypeptide may be contained within a composition that may, for example, be ingested orally in the form of a tablet, capsule or liquid. An alternative option for administrating Cyclic T14 (i.e. NBP14) would be to use a nasal spray, since peptide administration by nasal spray reaches the brain faster and more efficiently than oral or intravenous ways of administration (see http://memoryzine.com/2010/07/26/nose-sprays-cross-blood-brain-barrier-faster-and-safer/). Hence, compositions comprising cyclic polypeptides of the invention may be administered by inhalation (e.g. intranasally). As shown in Table 2, the cyclic peptide of the invention (NBP-14) was detected in the brain, showing that intranasal delivery of NBP-14 is effective in delivering NBP-14 to the brain. The inventors have shown that as much as 20% of the cyclic peptides of the invention can reach and get into the brain.

Compositions may also be formulated for topical use. For instance, creams or ointments may be applied to the skin, for example, adjacent the brain.

Preferably, the cyclic polypeptides of the invention are administered intranasally.

Cyclic polypeptides according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with cyclic polypeptides used according to the invention is required and which would normally require frequent administration (e.g. at least daily injection).

In a preferred embodiment, medicaments according to the invention may be administered to a subject by injection into the blood stream or directly into a site requiring treatment. For example, the medicament may be injected close to, or at least adjacent the brain. Injections may be intravenous (bolus or infusion) or subcutaneous (bolus or infusion), or intradermal (bolus or infusion).

It will be appreciated that the amount of the cyclic polypeptide that is required is determined by its biological activity and bioavailability, which in turn depends on the mode of administration, the physiochemical properties of the cyclic polypeptide and whether it is being used as a monotherapy or in a combined therapy. The frequency of administration will also be influenced by the half-life of the cyclic polypeptide within or on the subject being treated. Optimal dosages to be administered may be determined by those skilled in the art, and will vary with the particular cyclic polypeptide in use, the strength of the pharmaceutical composition, and the mode of administration. Additional factors depending on the particular subject being treated will result in a need to adjust dosages, including subject age, weight, gender, diet, and time of administration.

Generally, a daily dose of between 0.001μ.g/kg of body weight and 10mg/kg of body weight, or between 0.01μg/kg of body weight and 1mg/kg of body weight, of the cyclic polypeptide according to the invention may be used for treating, ameliorating, or preventing Down's Syndrome, depending upon which cyclic polypeptide is used.

The cyclic polypeptide may be administered before, during or after onset of symptoms associated with Down's syndrome. Daily doses may be given as a single administration (e.g. a single daily application). Alternatively, the cyclic polypeptide may require administration twice or more times during a day. As an example, cyclic polypeptides may be administered as two (or more) daily doses of between 0.07 lag and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of cyclic polypeptide according to the invention to a patient without the need to administer repeated doses.

Known procedures, such as those conventionally employed by the pharmaceutical industry (e.g. in vivo experimentation, clinical trials, etc.), may be used to form specific formulations of the cyclic polypeptide according to the invention and precise therapeutic regimes (such as daily doses of the agents and the frequency of administration). The inventors believe that they are the first to suggest a Down's syndrome treatment composition, based on the use of a cyclic polypeptide of the invention.

Hence, in a third aspect of the invention, there is provided a Down's syndrome treatment pharmaceutical composition comprising a therapeutically effective amount of the cyclic polypeptide, derivative or analogue thereof comprising an amino acid sequence derived from the C-terminus of acetylcholinesterase (AChE), or a truncation thereof, and a pharmaceutically acceptable vehicle.

The invention also provides in a fourth aspect, a process for making the Down's syndrome treatment composition according to the third aspect, the process comprising combining a therapeutically effective amount of the cyclic polypeptide, derivative or analogue thereof comprising an amino acid sequence derived from the C-terminus of acetylcholinesterase (AChE), or a truncation thereof, with a pharmaceutically acceptable vehicle.

The cyclic polypeptide, derivative or analogue thereof preferably comprises or consists of Cyclic T14 (i.e. NBP-14) as disclosed herein, i.e. SEQ ID No: 3.

A “subject” may be a vertebrate, mammal, or domestic animal. Hence, medicaments according to the invention may be used to treat any mammal, for example livestock (e.g. a horse), pets, or may be used in other veterinary applications. Most preferably, however, the subject is a human being.

A “therapeutically effective amount” of cyclic polypeptide is any amount which, when administered to a subject, is the amount of active agent that is needed to treat Down's syndrome, or produce the desired effect. The cyclic polypeptide, derivative or analogue thereof may be used as an adjuvant for the treatment of Down's syndrome. This means that lower doses of other treatments would be required.

For example, the therapeutically or cosmetically effective amount of cyclic polypeptide used may be from about 0.001 mg to about 800 mg, and preferably from about 0.01 mg to about 500 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, coatings, or tablet-disintegrating agents. The vehicle may also be an encapsulating material. In powders, the vehicle is a finely divided solid that is in admixture with the finely divided active agents according to the invention. In tablets, the active agent (i.e. the modulator) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention (the cyclic polypeptide) may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The cyclic polypeptide may be prepared as a sterile solid composition that may be dissolved or suspended at the time of administration using sterile water, saline, or other appropriate sterile injectable medium.

The cyclic polypeptide and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The cyclic polypeptide used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the sequence identified as SEQ ID No:1-4, and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence, which hybridizes to DNA sequences or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in SEQ ID No: 1-4.

Due to the degeneracy of the genetic code, it is clear that any nucleic acid sequence described herein could be varied or changed without substantially affecting the sequence of the protein encoded thereby, to provide a functional variant thereof

Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

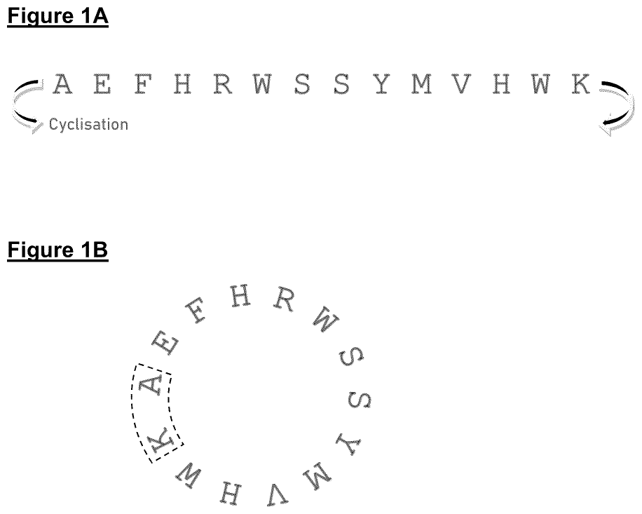

FIG. 1A shows the sequence of the linear peptide T14 (SEQ ID No:3) with the terminal Alanine (A) and Lysine (K) residues forming the cyclisation sites to form cyclic peptide, NBP-14. FIG. 1B shows the cyclic NBP-14 peptide in which the terminal Alanine and Lysine residues are linked together;

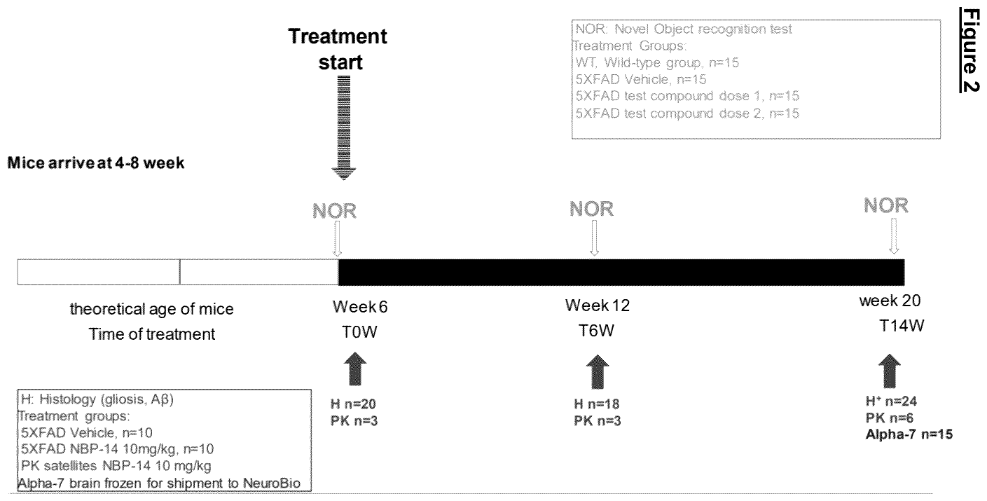

FIG. 2 shows a schematic representation of the efficacy study design for testing the effects of NBP-14 on mice.

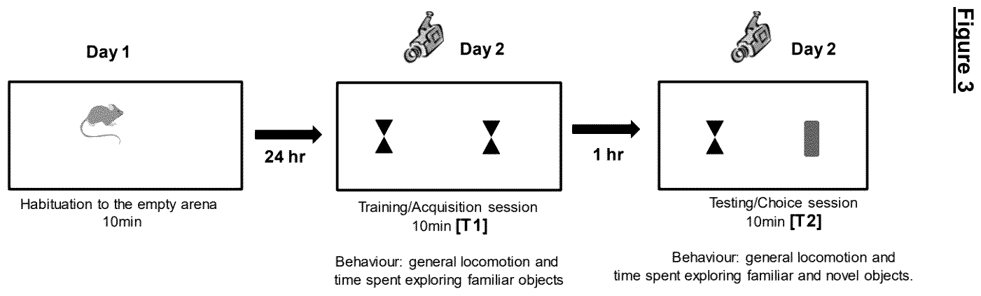

FIG. 3 shows a summary of the Novel Object Recognition Test used by the inventors to measure cognitive performance in mice.

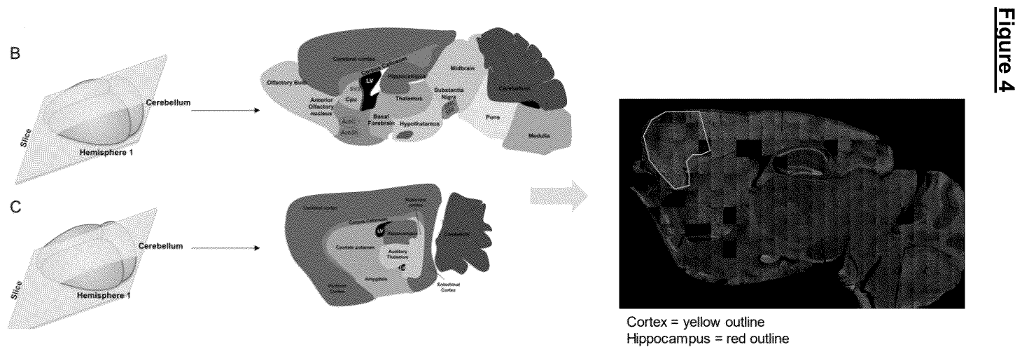

FIG. 4 shows a schematic representation of the tissue sampling method used by the inventors to identify markers of Alzheimer's Disease (AD) pathology.

FIG. 5 shows the results of the novel object recognition test, measuring the percentage of time taken by mice exploring familiar or novel objects a) in mice before the start of treatment, which shows that all groups of mice showed significant ability to discriminate the novel object vs familiar object. *p<0.05; **p<0.01 vs. familiar object, n=15 28. b) shows the results of the novel object recognition test in mice 6 weeks post treatment with NBP-14, and shows that only wild type mice spent significantly more time exploring the novel object compared to the familiar object. *p<0.05; **p<0.01 vs. familiar object, n=14 28. c) shows the results of the novel object recognition test in mice 14 weeks post treatment and that NBP-14 is able to reverse the decline. *p<0.05; **p<0.01 vs. familiar object, n=15 28.

FIG. 6 shows the recognition index of transgenic Tg-5XFAD mice. A recognition index above 50% reflects the ability of mice to explore an unfamiliar object (novel) than a recently presented object. NBP-14 was able to reverse a progressive reduction in cognitive performance in the 5XFAD mice.

FIG. 7 shows the effect of chronic NBP-14 treatment on grooming/sitting behaviours, as measured by behavioural scoring. A clear trend of a reduction in sitting behaviour was observed in vehicle-treated Tg-5XFAD mice and this was reversed with treatment of NBP-14.

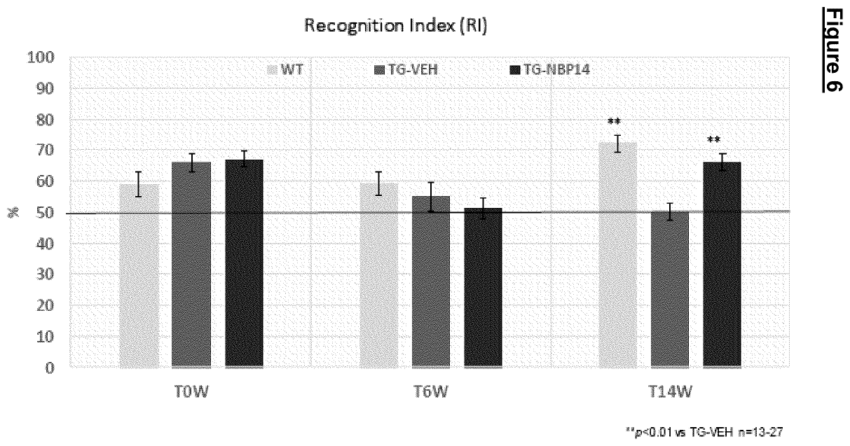

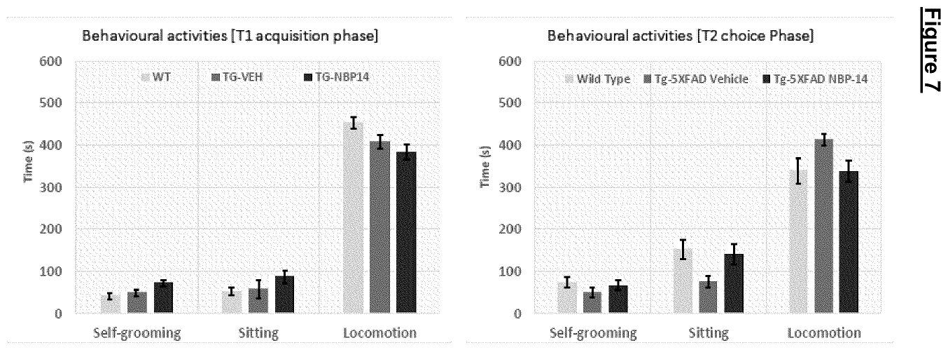

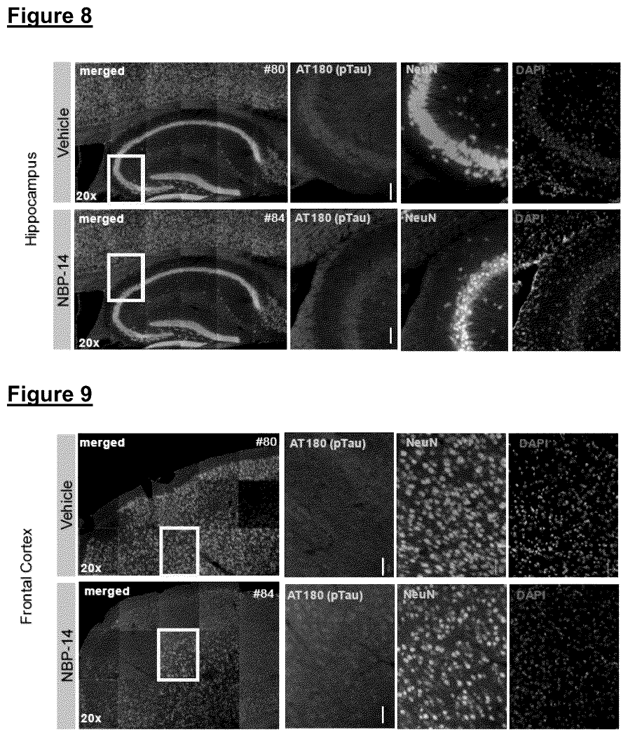

FIG. 8 shows immunostaining of Alzheimer's disease markers pTau and NeuN in the hippocampus of 5XFAD mice acutely treated with vehicle or NBP-14. Scale bar=50 μm.

FIG. 9 shows immunostaining of Alzheimer's disease markers pTau and NeuN in the frontal cortex of 5XFAD mice acutely treated with vehicle or NBP-14. Scale bar=50 μm.

FIG. 10 shows the image analysis strategy for quantifying the density of NeuN positive neurons. Only NeuN staining is quantified. AT180 staining is considered only background.

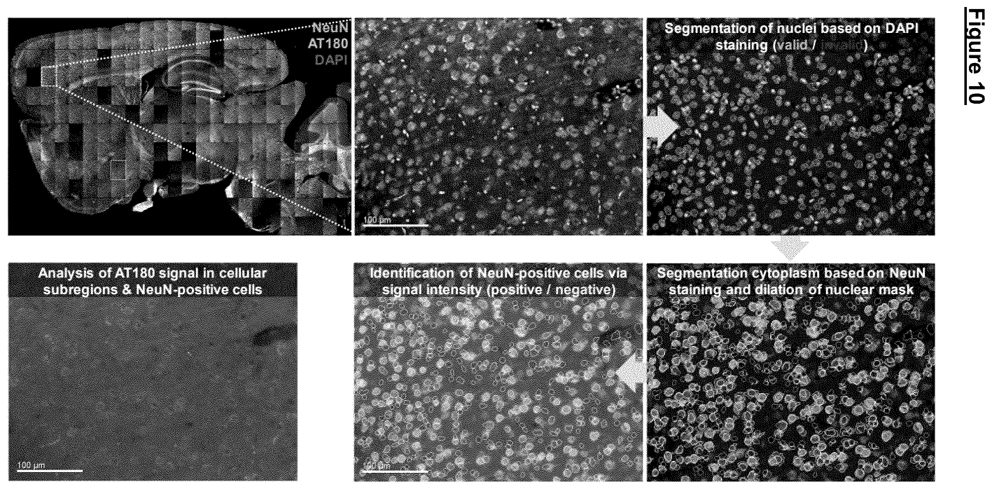

FIG. 11 shows that there is no difference in the density of NeuN positive neurons n the hippocampus or cortex of vehicle-treated and NBP-14-treated mice.

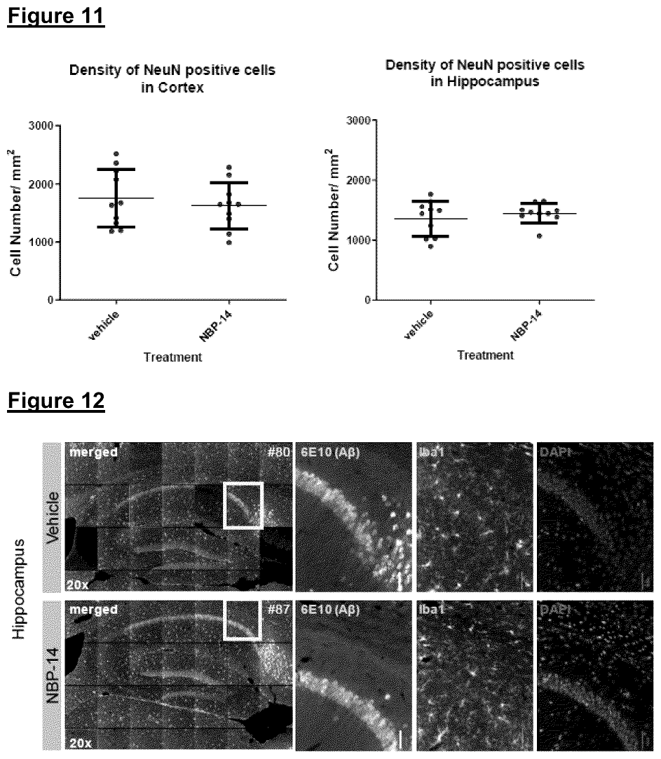

FIG. 12 shows immunostaining of β-amyloid (using the 6E10 antibody) in the hippocampus of 5XFAD mice acutely treated with vehicle or NBP-14. Scale bar=50 μm.

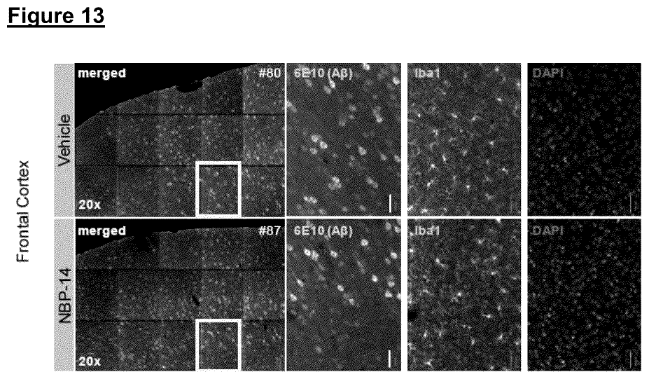

FIG. 13 shows immunostaining of β-amyloid (using the 6E10 antibody) and Iba1 (using the lba1 antibody) in the cortex of 5XFAD mice acutely treated with vehicle or NBP-14. Scale bar=50 μm.

FIG. 14 shows the image analysis strategy for quantifying the levels of 6E10 and Iba1 for detecting β-amyloid and lba1 respectively.

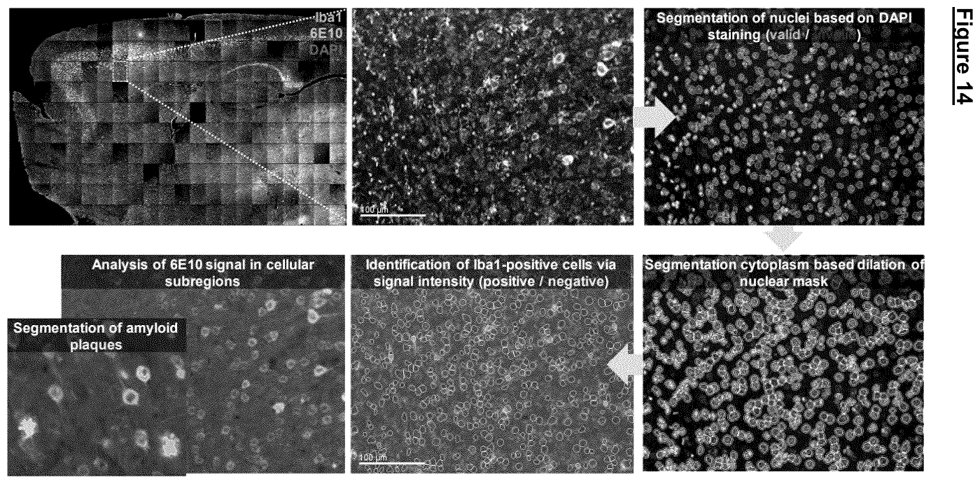

FIG. 15 shows the quantitative analysis of the differences between 6E10 and Iba1 levels in the hippocampus or cortex of mice treated acutely with vehicle or NBP-14. There were no significant differences observed.

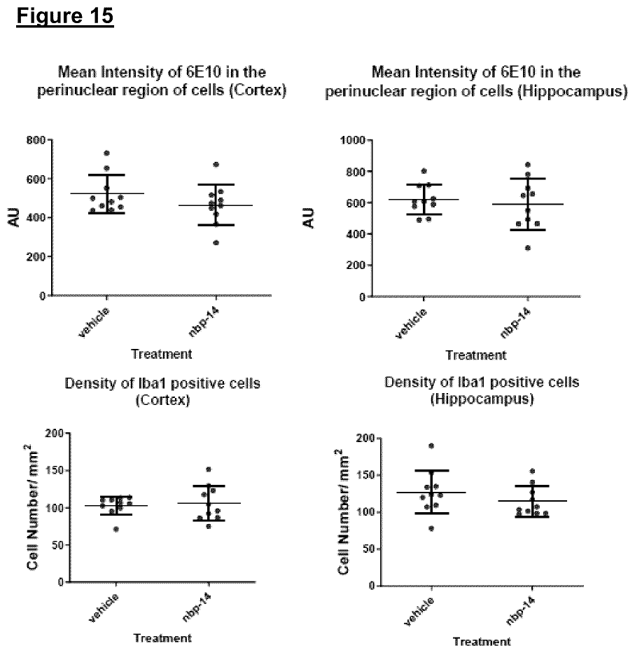

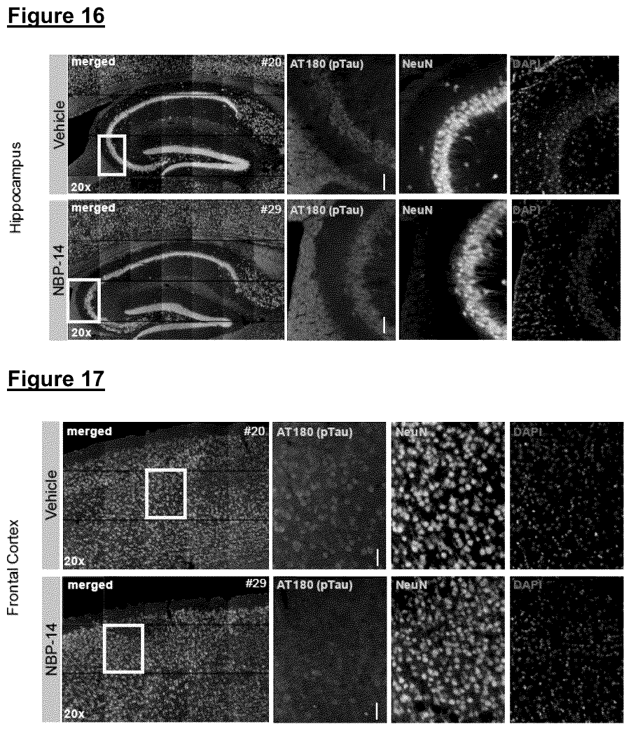

FIG. 16 shows immunostaining of Alzheimer's disease markers pTau and NeuN in the hippocampus of 5XFAD mice treated for 6 weeks with vehicle or NBP-14. Scale bar=50 μm.

FIG. 17 shows immunostaining of Alzheimer's disease markers pTau and NeuN in the cortex of 5XFAD mice treated for 6 weeks with vehicle or NBP-14. Scale bar=50 μm.

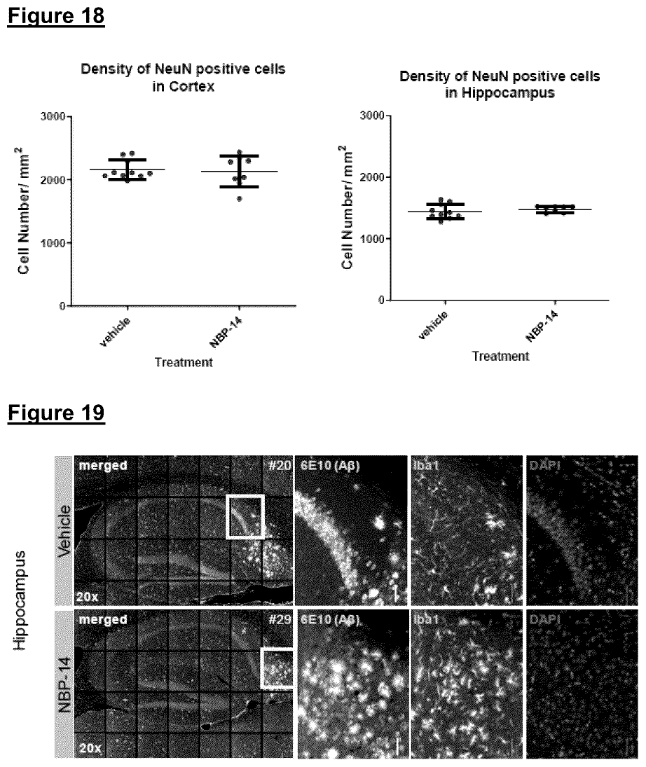

FIG. 18 shows the quantitative analysis of the density of NeuN positive neurons and that there is no difference in the density of NeuN positive neurons in the hippocampus or cortex of vehicle-treated and NBP-14-treated mice.

FIG. 19 shows immunostaining of β-amyloid (6E10 antibody) in the hippocampus of 5XFAD mice treated with vehicle or NBP-14 for 6 weeks. Scale bar=50 μm.

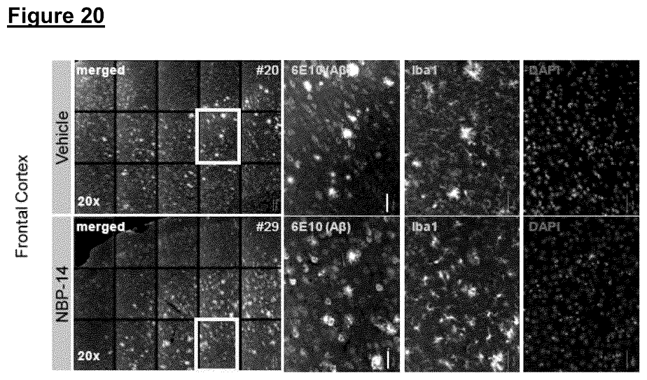

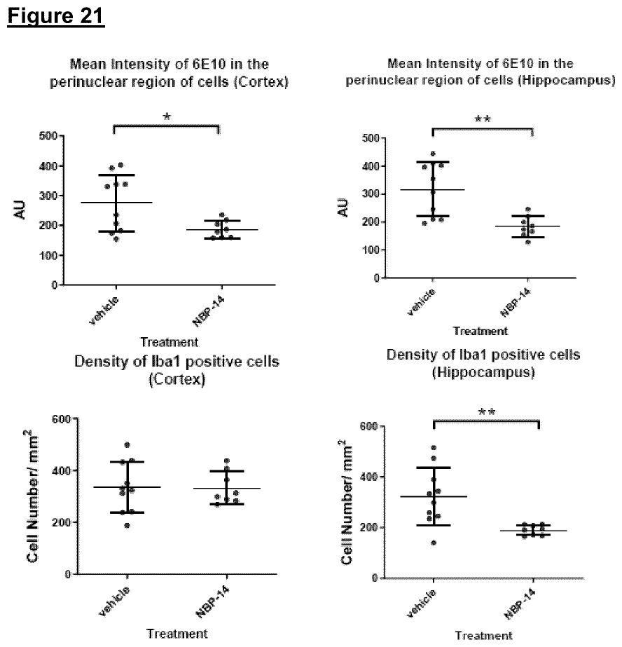

FIG. 20 shows immunostaining of β-amyloid (6E10 antibody) and Iba1 in the cortex of 5XFAD mice treated with vehicle or NBP-14 for 6 weeks. Scale bar=50 nm.

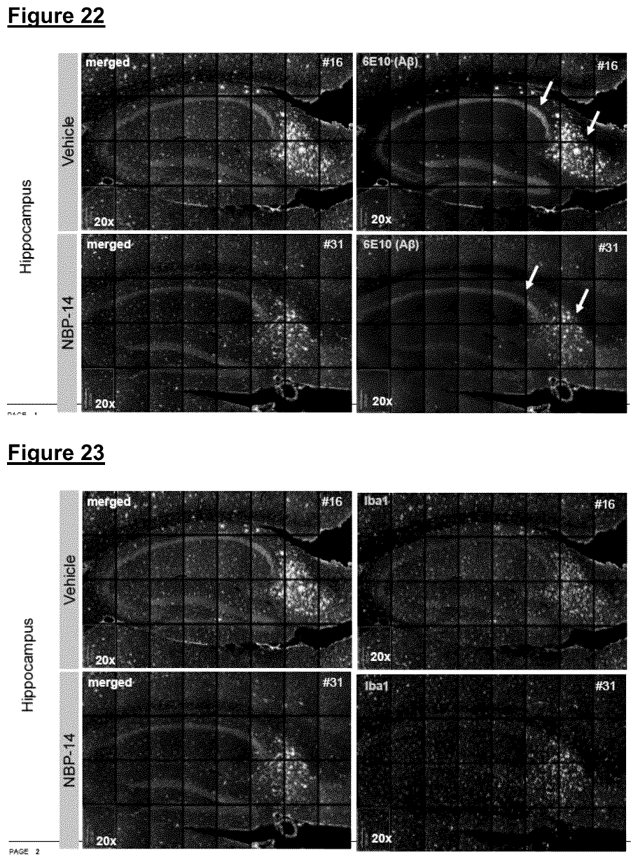

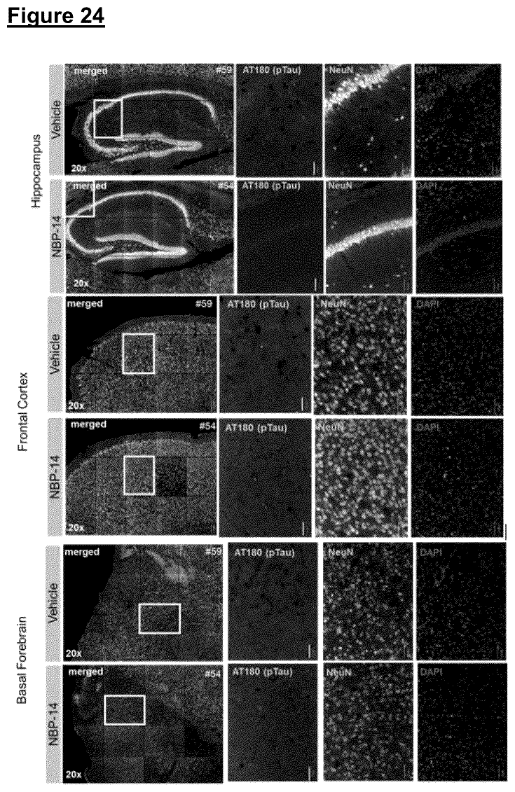

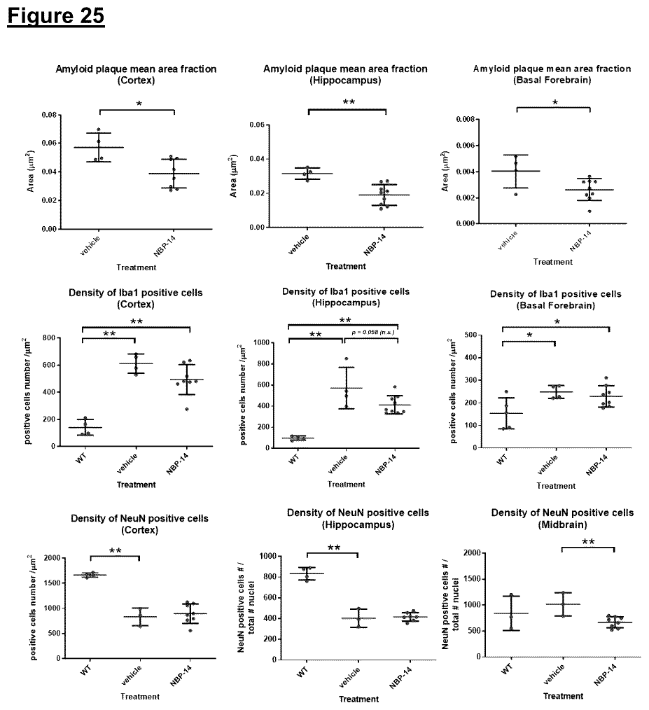

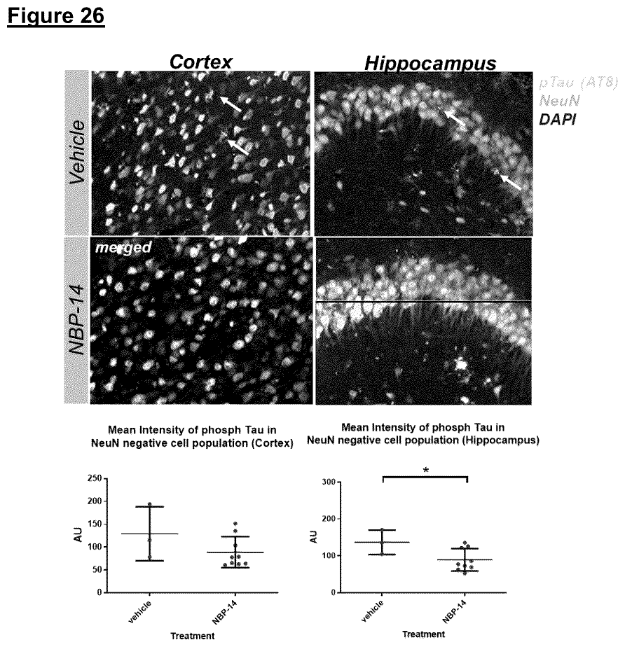

FIG. 21 shows the quantitative analysis of the differences between 6E10 antibody and Iba1 antibody levels in the hippocampus or cortex of mice treated for 6 weeks with vehicle or NBP-14. A significant decrease in the mean intensity of 6E10 (to bind to Aβ) is observed in the cortex and hippocampus of mice after 6 weeks of treatment with NBP-14 compared to vehicle and a significant difference of Ibal-positive cells is observed in the hippocampus after 6 weeks of treatment with NBP-14 compared to vehicle. Statistical analysis was performed using an unpaired t test. P≤0.05=*; P<0.005=**.

FIG. 22 shows additional, individual mouse data showing immunostaining of β-amyloid (6E10 antibody) in the hippocampus of SXFAD mice treated with vehicle or NBP-14 for 6 weeks. Scale bar=50 nm.

FIG. 23 shows additional, individual mouse data showing immunostaining of β-amyloid (6E10 antibody) in the cortex of 5XFAD mice treated with vehicle or NBP-14 for 6 weeks. Scale bar=50 μm.

FIG. 24 shows the effect of chronic NBP-14 treatment (T14W) on histological markers of AD pathology (pTau, NeuN) in 5XFAD mice. The Figure shows immunohistochemical staining of brain sections from 5XFAD Tg mice following chronic treatment with NBP-14 or vehicle. No pTau (gold) can be detected in the hippocampus (A) cortex (B) or basal forebrain (C). Neurons are detected with NeuN (green). Nuclei are detected with DAPI (blue). White boxes are shown as enlarged images to the right of the merged overview image.

FIG. 25 shows quantitative analysis of effects on histological markers of AD pathology (6E10, Iba1, NeuN) following chronic NBP-14 treatment (T14W) in 5XFAD mice. The Figure shows quantification of IHC markers of AD pathology in different brain regions of 5XAFD mice after chronic treatment with NBP-14 or vehicle for 14 weeks. Extracellular Af3 intensity (A), Density of Iba1 positive cells (B) and density of NeuN positive cells (C). Statistical analysis was performed using an unpaired t test. *p<0.05; **p<0.01 vs. Vehicle; n=4, NBP-14, n=8; 6 sections per animal.

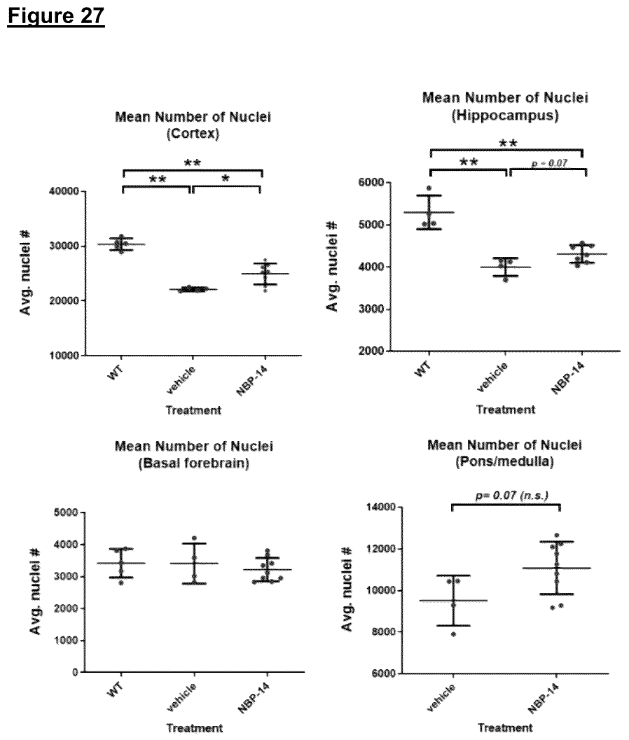

FIG. 26 shows the effect of chronic NBP-14 treatment (T14W) on histological markers of AD pathology (pTau, AT8) in 5XFAD mice. The Figure shows immunohistochemical staining of sections from 5XFAD Tg mice following chronic treated with NBP-14 or vehicle for 14 weeks. Very low levels of pTau (gold) detected with AT8 (pS202/pT205) is observed in non neuronal cells (NeuN negative) in the cortex and hippocampus of vehicle treated 5XFAD mice (white arrows) but not in mice treated with NBP-14 (A). Neurons are detected with NeuN (green). Nuclei are detected with DAPI (blue). Quantitative analysis of AT8 IHC signal in NeuN negative cell population (B).

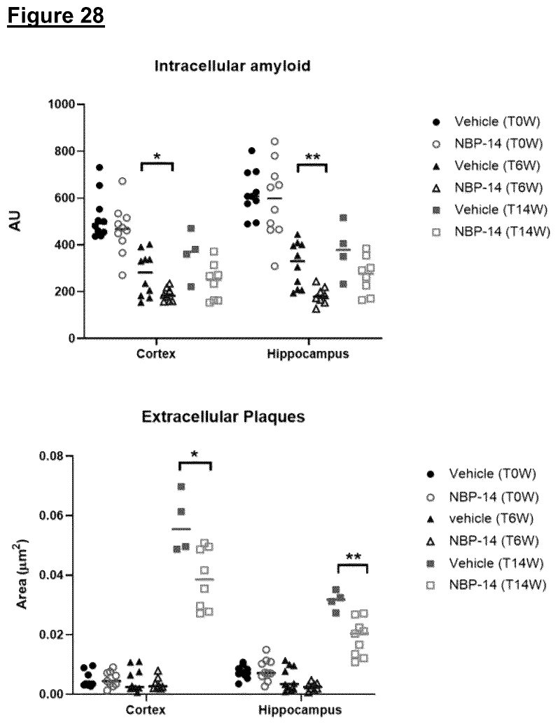

FIG. 27 shows quantitative analysis of effects on histological markers of AD pathology following chronic NBP-14 treatment (T14W) in 5XFAD mice. The Figure shows quantification of total number of nuclei in different brain regions of 5XAFD mice after chronic treatment with NBP-14 or vehicle for 14 weeks. Extracellular Al3 intensity (A), density of Iba1 positive cells (B) and density of NeuN positive cells (C). Statistical analysis was performed using an unpaired t test. *p<0.05; **p<0.01 vs. Vehicle; n=4, NBP-14, n=8; 6 sections per animal.

FIG. 28 shows quantitative analysis of effects on histological markers of AD pathology following chronic NBP-14 treatment (T14W) in 5XFAD mice. The Figure shows a comparison of the levels of intracellular amyloid and extracellular plaque deposits in the three different treatment groups of 5XAFD mice after acute or chronic treatment with NBP-14 or vehicle. Statistical analysis was performed using an unpaired t test. *p<0.05; **p<0.01 vs. vehicle.

### EXAMPLES

Rationale

The inventors utilised the transgenic mouse model, TG-5XFAD, which develops β-amyloid plaques and displays a phenotype associated with cognitive decline which is similar to early onset dementia that is observed in Down's syndrome. They investigated the ability of cyclic peptides derived from the C-terminus of acetylcholinesterase to reduce β-amyloid plaque formation in the mouse model and reverse symptoms associated with early onset dementia, and therefore present a novel therapy for Down's syndrome.

Materials and Methods

Cyclisation of Peptides

Three techniques were used to achieve cyclization of linear peptides described herein, i.e. side-chain-to-side-chain, side-chain-to-backbone, and head-to-tail (C-terminus to N-terminus) cyclization. Head-to-tail cyclization has been investigated extensively, and can involve directed Cys-Cys disulphide cyclization (up to two per molecule). Careful monitoring of the reaction ensures 100% cyclization. Two general approaches are used for synthesis: (1) classical solution-phase linear peptide cyclization under high dilution conditions; and (2) resin-based cyclization. Two distinct protocols were employed in the solid phase synthesis (1):
(a) The on-resin cyclization of a peptide anchored via a side-chain functional group, such as imidazole, 3 acid, 4 amine' or alcohol, was carried out. The peptide was orthogonally protected as an ester at the C-terminus, and the peptide was then assembled through regular Boc or Fmoc synthesis followed by saponification, cyclization and cleavage.
(b) Another protocol that was used was the cyclization cleavage approach, in which the cyclic peptide was synthesized by cyclization after step-wise linear peptide synthesis. One advantage of this method is that the side-chain does not need to be anchored, making the approach more general than (a). (Christopher J. White and Andrei K. Yudin (2011) Nature Chemistry 3; Valero et al (1999) J Peptide Res. 53, 76-67; Lihu Yang and Greg Morriello (1999) Tetrahedron Letters 40, 8197-8200; Parvesh Wadhwani et al (2006) J. Org. Chem. 71, 55-61).

Study Design of Preclinical Translational Pharmacology Studies

The study design is summarised in FIG. 2.

Animals

Female transgenic 5XFAD mice (B6SJL Tg(APPSwF1Lon,PSEN1*M146L*L286V) 6799Vas/ Mmjax) from Jackson Labs. Range of age: 5 8 weeks.

Female Wild Type mice (B6SJL_genetic background C57BL/6×SJL) from Jackson

Labs. Age: 4 weeks.

Treatment

Intranasal (IN, nose to brain) twice a week for 14 weeks (Volume of administration 10 μL/.

Groups of Treatment

Group 1 WT mice (only for NOR test);

Group 2 TG VEH: 5xFAD mice treated with vehicle of formulation (0.9% NaCl);

Group 3 TG NBP14: 5xFAD mice treated with NBP14 at the dose of 10 mg/kg; and

Group 4 TG NBP14: 5xFAD mice treated with NBP14 at the dose of 30 mg/kg (10 mg/kg starting from 2nd week of treatment due to relevant clinical sign at 30 mg/kg).

Readouts

Assessment of Novel Object Recognition (NOR) test at the following time points:
T0W, basal NOR behaviour immediately before starting of the experiment.
T6W, 6 weeks after starting of treatment.
T14W, 14 weeks after starting of treatment.

Assessment of Immunohistochemistry over the duration of the study at the same time points of NOR testing in:
Satellite group of mice at T0W and at T6W.
Mice from NOR test at T14W.

Assessment of PK profile for NBP14, 10mg/kg, to allow PK/PD correlations
Satellite group of mice at T0W and T6W.
Mice from NOR test at T14W.

Study Design of PK Assessment of NBP-14

Subjects

Group 3 TG NBP14: 5XFAD mice (n=3 for each PK) treated with NBP14 at the dose 10 mg/kg.

PK assessment time points:

After 1 single treatment at the start of treatment (T0W) in a satellite group of mice.

After 6 weeks of treatment (T6W) in a satellite group of mice. The day of PK profile mice were treated with NBP14.

After 14 weeks of treatment (14W) in 2 group of mice from NOR cohorts.

On the day of PK study, mice were treated with NBP14 at 10mg/kg (IN) and blood/brain collected at 30 minutes after treatment.

An additional group of mice was subjected to blood/brain collection for NBP14 exposure without treatment on the same day at the terminal time point, after 14 weeks of treatment, to evaluate an eventual accumulation of test compound (table 1).

Assessment of NOR test for cognitive read out study design

Subjects

Group 1 WT mice (n=15) used as control animal during the experimental procedure. No subject to treatment.

Group 2 TG VEH: 5XFAD mice (n=14) treated with vehicle of formulation (saline).

Group 3 TG NBP14: 5XFAD mice (n=28) treated with NBP14 at the dose 10 mg/kg.

NOR behavioural assessment time points:
Immediately before start of treatment (T0W)
6 weeks after start of treatment (T6W)
14 weeks after start of treatment (T14W)

Additional Scoring

Grooming/sitting/locomotion was measured on the mice subjected to NOR testing [i.e., Wild Type data included at 14 weeks post treatment].

At the end of the study and following the NOR procedure Tg 5XFAD mice were used for both PK (n=6 TG NPB14) and Histology (n=9 TG VEH; n=15 TG NPB14) and

Apha 7 assessment (n=5 TG VEH; n=10 TG NPB14).

Novel Object Recognition Test

The object recognition test is summarised in FIG. 3.

Behavioural Measure (Observer XT®)

Behaviour recorded on video for subsequent scoring for the object exploration. Object investigation has been defined as directing the nose towards the object (i.e., sniffing or touching with the nose) at a distance of 2 cm or less. Climbing and sitting on the objects is not considered to be object examination.

A criterion of minimal level of object exploration was used in the study to exclude animals with naturally low levels of spontaneous exploration: mice having a minimal level of object exploration of lOs during the test trail will be included in the study.

Results were expressed as the total time spent (seconds) by animal towards the objects. The recognition index (RI) was also calculated as follows: (time exploring the novel object)/(time exploring novel+familiar)*100.

Histology

Subjects

Group 2 TG-VEH: 5xFAD mice treated with vehicle of formulation (saline).

Group 3 TG-NBP14: 5xFAD mice treated with NBP-14 at the dose 10 mg/kg.

Histological Time-Points:
After 1 single treatment at the start of treatment, in a satellite group of mice (T0W).
After 6 weeks of treatment (T6W) in a satellite group of mice.
After 14 weeks of treatment (14W) in a group of mice from NOR cohorts (analysis ongoing).

Histological Analysis

Brain samples from all Tg-5XFAD mice were fixed, cryosectioned and immunostained for detection of amyloid, phosphorylated Tau and gliosis using the antibodies listed in Table 1.

TABLE 1
Antibodies (in the right hand column) used for immunohistological
analysis of brain samples of transgenic Tg 5XFAD study mice

| Gliosis | Activated microglia | Lba1 |
|---|---|---|
| Aβ Plaque | Beta amyloid | 6E10 |
| Tau | Phosphorylated tau | AT180 |
| Gliosis | Activated microglia | Lba1 |
| Cell loss | Neuronal cell count | NeuN |

Tissue Sampling Method

As shown in FIG. 4, cryosectioning of fixed and embedded brain samples was performed using a cryostat along the sagittal plane starting at the midline.

Serial sections were collected and every sixth section starting at the midline were immunostained for markers of AD pathology. A total of six sections per animal were used for quantitative analysis.

### Example 1

### Cyclic T14 (i.e. “NBP-14”)

The ‘tailed’ acetylcholinesterase (T-AChE) is expressed at synapses and the inventors have previously identified two peptides that could be cleaved from its C-terminus, one referred to as “T14” (14 amino acids long), within the other which is known as “T30” amino acids long). The amino acid sequence of the linear peptide, T14, is AEFHRWSSYMVHWK [SEQ ID No:3]. The amino acid sequence of the linear peptide, T30, is KAEFHRWSSYMVHWKNQFDHYSKQDRCSDL [SEQ ID No:2]. Another peptide referred to as “T15” corresponds to the last 15 amino acid residues of SEQ ID No: 1, i.e. NQFDHYSKQDRCSDL [SEQ ID No: 4].

The AChE C-terminal peptide “T14”' has been identified as being the salient part of the AChE molecule responsible for its range of non-hydrolytic actions. The synthetic 14 amino acids peptide analogue (i.e. “T14”), and subsequently the larger, more stable, and more potent amino acid sequence in which it is embedded (i.e. “T30”) display actions comparable to those reported for ‘non-cholinergic’ AChE.

Referring first to FIG. 1A, there is shown the 14 amino acid long cyclic T14 peptide (i.e. “NBP-14”). The cyclic peptide, NBP-14, has been cyclised via the terminal Alanine (A) and Lysine (K) residues, and is shown in FIG. 1B. Cyclisation can be achieved by several different means. For example, Genosphere Biotechnologies (France) performed the cyclisation of T14 by transforming the linear peptide into an N-terminal to C-terminal lactam. Cyclisation of T14 to create cyclic NBP-14 brings together both ends, i.e. HWK-AEF.

### Example 2

### Assessment of Blood/Brain Exposure to NBP-14

The inventors measured the concentration of nasally applied NBP-14 in the blood and the brain of mice, to determine whether NBP-14 was capable of crossing the blood brain barrier when applied intranasally.

As shown in Table 2, after both 6 weeks and 14 weeks of treatment, NBP-14 was detected in the brain, showing that intranasal delivery of NBP-14 is effective in delivering NBP-14 to the brain. No NBP-14 was detected in a group mice treated for 14 weeks with NBP-14 but not treated on the day of blood/brain collection, which indicates that there is no accumulation of the compound.

TABLE 2
Blood/brain exposure to NBP-14

| Animal | Treatment | Batch | Blood | Brain | |
|---|---|---|---|---|---|
| Number | Time Point | Number | Concentration ng/mL or ng/g) | | BB Ratio |
| Sampling 5 Mar. 2020 (sampling 30 min after administration) | | | | | |
| M39 | T = 0 | 114352 | 44.5 | bql | NC |
| M40 | | | 42.4 | bql | NC |
| M41 | | | 38.9 | bql | NC |
| Sampling 5 Mar. 2020 (sampling 30 min after administration) | | | | | |
| M36 | T = 6W | 114352 | 204 | 19.3 | 0.095 |
| M37 | [intermediate] | | 584 | 16.8 | 0.029 |
| M38 | | | 251 | blq | NC |
| Sampling 6 May 2020 (sampling 30 min after administration) | | | | | |
| M6 | T = 14W | | 202 | 53.0 | 0.26 |
| M7 | [terminal] | 115250 | 292 | 60.4 | 0.21 |
| M8 | | | 171 | 51.4 | 0.30 |
| Sampling 21 May 2020 (sampling 30 min after administration) | | | | | |
| M47 | T = 14W | no treated | bql | bql | NC |
| M48 | [terminal] | last day | bql | bql | NC |
| M49 | | | bql | bql | NC |

BQL: below quantification limit
NC: not calculated

### Example 3

### Novel Object Recognition Test for Cognitive Readout

NBP-14 Treatment on Object Exploration Time

The inventors utilised a “novel object recognition” test, which measures the difference of the time spent exploring an unknown (i.e. novel) object versus a known or familiar object to determine the ability of mice to discriminate between novel and familiar objections that can be used as in indication of memory. The inventors used this test to determine the ability of NBP-14 to reverse memory decline in the transgenic mouse, Tg 5XFAD, predisposed to develop amyloid precursor protein (APP) and therefore amyloid plaques, and ultimately, dementia.

As shown in FIG. 5a, the inventors first the confirmed that Tg 5XFAD mice do not display cognitive deficits in the range of age of 5-8 weeks and so no difference was observed between the wild-type, Tg 5XFAD vehicle treated and Tg 5XFAD NBP-14 treated mice.

Referring now to FIG. 5b, after 6 weeks (estimate age of mice was about 12 weeks), wild type mice spent more time exploring the novel object versus familiar object. 5XFAD mice treated with vehicle (TG-VEH, n=14) at 6 weeks after treatment (estimate age of mice 11-14 weeks) showed no statistical difference in the time exploring the novel object versus familiar object, although high variability was observed in these mice. 5XFAD mice treated with NBP14 (TG-NBP14, n=28) at 6 weeks after treatment (estimate age of mice 11-14 weeks) also showed no significant difference in the time exploring the novel versus familiar object, as shown in FIG. 5b.

As shown in FIG. 5c, however, the inventor observed a statistically significant difference in the time spent exploring novel object versus familiar object in the wild type mice (n=15) after 14 weeks (estimate age of mice was 22 weeks). No statistical significant difference was observed on the time spent exploring novel object versus familiar object in 5XFAD mice treated with vehicle (TG-VEH, n=14) 14 weeks after treatment (estimate age of mice 19-22 weeks). However, most surprisingly, a statistically significant difference on the time spent exploring novel object versus familiar object was clearly observed in the 5XFAD mice treated with NBP14 (TG-NBP14 group, n=27) 14 weeks after treatment (estimate age of mice 19-22 weeks).

Thus, these data clearly and surprising show that NBP-14 has a significant protective effect on cognitive decline in the transgenic mice otherwise predisposed to develop dementia.

Effects of Chronic NBP14 Treatment (10 mg/kg) in the Recognition Index in Tg 5XFAD Mice

The inventors then determined the recognition index of mice. A recognition index above 50% reflects the ability of mice to explore an unfamiliar object (novel) than a recently presented object.

As shown in FIG. 6, the study revealed a progressive reduction in cognitive performance in the transgenic 5XFAD mice as indicated by recognition index (baseline vs 14 weeks) confirming the validity of the NOR procedure to reveal cognitive deficits in this mouse model of AD.

A statistical significant difference of RI was observed in the WT mice (n=15, age 22 weeks) and 5xFAD mice treated with NBP14, i.e. TG=NBP14, (n=27, age 19-22 weeks) compared to vehicle-treated 5xFAD TG-VEH mice (n=13, age 19-22 weeks) group at the terminal time point. This surprisingly shows that NBP-14 protects against cognitive decline, especially after 14 weeks post treatment.

Effect of Chronic NBP-14 Treatment on Grooming/Sitting Behaviours

Behavioural scoring was assessed during the T1 (familiar) and T2 (novel) phase of NOR procedure (over 10 min each phase). As shown in FIG. 7, a clear trend of a reduction in sitting behaviour was observed in vehicle-treated Tg-5XFAD mice and this was surprisingly reversed with treatment of NBP-14.

Conclusions

Baseline cognitive performance obtained in all groups of mice confirm the validity of the selected protocol to assess NOR in the mouse and indicate that cognitive function in 5XFAD mice of 6-8 weeks of age is similar to WT mice.

The study revealed a progressive reduction in cognitive performance in the 5XFAD mice as indicated by recognition index (baseline vs 14 weeks) confirming the validity of the NOR procedure to reveal cognitive deficits in this mouse model of AD.

These findings are in agreement with literature reports indicating that 5XFAD mice start to show cognitive function abnormalities between 4-6 months of age (Giannoni et al., 24 Dec2013, Front. Aging NeuroSci.; Creighton, et al., Nature, Scientific Reports, 2019, 9:57).

At the 6 week time point of NOR testing, there were no statistically significant differences in cognitive performance of NBP-14-treated 5XFAD mice compared to 5XFAD vehicle-treated mice age of mice at this stage (i.e. 12-14 weeks).

At the 14 week time point of NOR testing, an impaired ability to discriminate between the familiar and novel objects is observed in 5XFAD mice treated with vehicle as shown by recognition index (age of mice at this stage 19-22 weeks). However, 5XFAD mice treated with NPB-14 did not demonstrate an impaired recognition index suggesting a protective effect on cognitive decline by NBP-14 in this experimental condition (age of mice at this stage 19-22 weeks).

In addition to the primary cognitive readout on NOR, an additional analysis was utilised to assess for qualitative changes on general behaviour over the study period. These data show the clear trend for reduction in sitting behaviour in Tg-5XFAD vehicle-treated mice, which was not observed in WT mice or 5XFAD-NPB-14-treated mice.

Overall, the cognitive performance and general behaviours of NPB-14-treated 5XFAD and untreated WT mice at the 14 week time point was very similar, and shows that NBP-14 is surprisingly able to reverse the cognitive decline observed in Tg-5XFAD mice.

### Example 4

### Effect of NBP14 Treatment on Markers of Pathology in Transgenic 5XFAD Mice

Having shown the ability of NBP-14 to reverse the cognitive decline observed in Tg-5XFAD mice as discussed above, the inventors then sought to determine the structural or physiological changes occurring in the brains of mice treated with NBP-14. The inventors utilised histological staining of the brain to determine the changes of various brain-located markers (i.e. phosphorylated Tau, NeuN, (3-amyloid, and Ibal) that are associated with cognitive decline in both 5XFAD mice and also mice that had been treated with NBP-14. These biomarkers were measured after acute treatment with NBP-14 and also six weeks after treatment.

Acute Treatment with NBP-14

As shown in FIGS. 8 and 9, no specific intracellular phosphorylated Tau immunoreactivity was detected with the antibody, AT180, or observed in either the hippocampus (FIG. 9) or the cortex (FIG. 10) of vehicle-treated and NBP-14-treated 5XFAD mice. Only non-specific background staining was observed.

In addition, as shown in FIGS. 10 and 11, no differences in the total number or density of NeuN positive neurons were observed between the cortex or in the hippocampus of mice treated acutely with NBP-14 compared to the vehicle.

As shown in FIGS. 12 to 15, intracellular β-amyloid (Aβ) is observed in pyramidal neurons in CAl of the hippocampus and in the subiculum. A few small extracellular plaque deposits are observed in the subiculum of both vehicle-treated and NBP14-treated 5XFAD mice. No differences in the mean intensity of the antibody, 6E10, which binds to Aβ were observed by a visual inspection in the cortex or in the hippocampus of mice treated acutely with NBP-14 compared to vehicle. Also, there were no differences in the total number or density of Ibal-positive cells observed by visual inspection in the cortex or in the hippocampus of mice treated acutely with NBP-14 compared to vehicle. These data align with the blood/brain measurements of NBP-14, where no NBP-14 was observed in the brain after acute treatment.

6 Weeks Treatment with NBP-14

As shown in FIGS. 16 and 17, no specific intracellular phosphorylated Tau immunoreactivity was detected or observed with the antibody, AT180, in the hippocampus or the cortex of either vehicle-treated and NBP-14-treated 5XFAD mice. In addition, as shown in FIG. 18, there were no differences in the total number or density of NeuN positive neurons observed in the cortex or in the hippocampus of mice after 6 weeks of treatment with NBP-14 compared to vehicle.

However, surprisingly, the inventors observed a significant decrease in the mean intensity of β-amyloid using the antibody, 6E10, in the cortex and hippocampus of mice after 6 weeks of treatment with NBP-14 when compared to vehicle, as shown in FIGS. 19 to 24.

For example, FIG. 19 shows immunostaining of I3-amyloid using the 6E10 antibody in the hippocampus of 5XFAD mice treated with vehicle or NBP-14 for 6 weeks, and FIG. 20 shows immunostaining of β-amyloid (6E10 antibody) and Iba1 in the cortex of 5XFAD mice treated with vehicle or NBP-14 for 6 weeks. As can be seen, in both cases there is a significant (respectively P<0.005 and P<0.05) decrease in intracellular amyloid relative to vehicle-treated controls, accompanied in the hippocampus by a significant (P<0.005) decrease in gliosis.

FIG. 21 shows the quantitative analysis of the differences between 6E10 antibody and Iba1 antibody levels in the hippocampus or cortex of mice treated for 6 weeks with vehicle or NBP-14. As can be seen, a significant decrease in the mean intensity of 6E10 (to bind to A(3) is observed in the cortex and hippocampus of mice after 6 weeks of treatment with NBP-14 compared to vehicle and a significant difference of Iba1-positive cells is observed in the hippocampus after 6 weeks of treatment with NBP-14 compared to vehicle.

FIGS. 22 and 23 show additional, individual mouse data showing immunostaining of β-amyloid (6E10 antibody) in the hippocampus and cortex, respectively, of 5XFAD mice treated with vehicle or NBP-14 for 6 weeks. As can be seen, there is a marked decrease in both cases in signal, in the NBP-14-treated mouse compared to a vehicle-treated counterpart.

14 Weeks Treatment with NBP-14

FIG. 24 shows immunohistochemical staining of brain sections from 5XFAD Tg mice following chronic treatment with NBP-14 or vehicle after 14 weeks. As is shown, no pTau (gold) was detected in the hippocampus (A) cortex (B) or basal forebrain (C).

Similarly, FIG. 26 shows immunohistochemical staining of sections from 5XFAD Tg mice following chronic treated with NBP-14 or vehicle for 14 weeks. Very low levels of pTau (gold) detected with AT8 (pS202/pT205) is observed in non-neuronal cells (NeuN negative) in the cortex and hippocampus of vehicle treated 5XFAD mice (white arrors) but not in mice treated with NBP-14 (A).

FIG. 25 shows quantitative analysis results of amyloid, gliosis and cell number in different brain regions of SXAFD mice after chronic treatment with NBP-14 or vehicle for 14 weeks. Extracellular Aβ intensity (A), Density of Iba1 positive cells (B) and density of NeuN positive cells (C) are shown.

FIG. 27 shows the quantification of total number of nuclei in different brain regions of 5XAFD mice after chronic treatment with NBP-14 or vehicle for 14 weeks. Extracellular AP intensity (A), Density of Iba1 positive cells (B) and density of NeuN positive cells (C) are shown.

FIG. 28 shows the comparison of the levels of intracellular amyloid and extracellular plaque deposits in the three different treatment groups of 5XAFD mice after acute or chronic treatment with NBP-14 or vehicle.

Without wishing to be bound to any specific theory, these data show that NBP-14 is able to reduce the formation of β-amyloid plaques, and also reverse cognitive decline. Structural changes in the brain are observed at 6 weeks, prior to the phenotypic changes that were observed after 14 weeks of treatment. The inventors hypothesize that even more pronounced structural changes would have been observed beyond 14 weeks post treatment, passing a threshold that ensures cognitive decline is reversed.

### SUMMARY

Down's Syndrome can be characterized in middle age by an accumulation of brain amyloid, that would be a contributing factor to compromising quality of life and even survival. If an effective treatment could be given that reduces amyloid, it would have the potential for a beneficial effect on both cognition and/or lifespan.

As described herein, the inventors intranasally applied the cyclic peptide, NBP-14, to transgenic Tg-5XFAD mice, and observed a significant decrease in the intensity of intracellular β-amyloid in the hippocampus and cortex of these Tg-5XFAD mice treated with NBP-14 (compared to the vehicle control) over a 6 week period. At 14 weeks, the amyloid had accumulated outside of the cells to form plaques that were significantly reduced by NBP-14 in the cortex, hippocampus and basal forebrain compared to the vehicle-treated controls. The inventors also found that NBP-14 has a significant protective effect on cognitive decline in the transgenic Tg-5XFAD mice otherwise predisposed to develop dementia, and that NBP-14 reversed the cognitive decline that was observed in the transgenic mice to a level of performance that was comparable to a wild-type group. This work has therefore shown that cyclic peptides derived from the C-terminus of acetylcholinesterase reduce β-amyloid formation and protect from, and reverse, cognitive decline, thereby indicating that these cyclic peptides can be used in the effective treatment Down's syndrome.

1-16. (canceled)

17. A method of treating, ameliorating or preventing Down's syndrome, the method comprising, administering, or having administered, to a subject in need of such treatment, a therapeutically effective amount of a cyclic polypeptide, derivative or analogue thereof comprising an amino acid sequence derived from the C-terminus of acetylcholinesterase (AChE), or a truncation thereof.

18. The method according to claim 17, wherein the cyclic polypeptide, derivative or analogue thereof is capable of reducing and/or inhibiting β-amyloid plaque formation in a Down's syndrome person.

19. The method according to claim 18, wherein the plaque formation is inhibited in the person's hippocampus and/or cortex.

20. The method according to claim 17, wherein the cyclic polypeptide, derivative or analogue thereof is capable of reducing and/or inhibiting phosphorylated Tau (pTau) formation in a Down's syndrome person.

21. The method according to claim 20, wherein the phosphorylated Tau formation is inhibited in the person's hippocampus and/or cortex.

22. The method according to claim 17, wherein the cyclic polypeptide, derivative or analogue thereof is capable of reducing, inhibiting and/or reversing cognitive decline in a Down's syndrome person.

23. The method according to claim 17, wherein the cyclic polypeptide, derivative or analogue thereof is capable of reducing, inhibiting and/or reversing dementia in a Down's syndrome person, preferably early onset dementia in a Down's syndrome person.

24. The method according to claim 17, wherein the cyclic polypeptide, derivative or analogue thereof is capable of reducing, inhibiting and/or reversing cognitive decline or dementia in a Down's syndrome person who is in their 20's, 30's, 40's, 50's, 60's or 70's.

25. The method according to claim 17, wherein the acetylcholinesterase comprises an amino acid sequence substantially as set out in SEQ ID No:1, or a variant or fragment thereof.

26. The method according to claim 17, wherein the cyclic polypeptide, derivative or analogue thereof, comprises between 4 and 50 amino acid residues, or between 6 and 40 amino acids, or between 8 and 30 amino acid residues.

27. The method according to claim 17, wherein the cyclic polypeptide, derivative or analogue thereof, comprises between 6 and 25 amino acid residues, or between 7 and 20 amino acid residues, or between 8 and 15 amino acid residues.

28. The method according to claim 17, wherein the cyclic polypeptide, derivative or analogue thereof comprises cyclic SEQ ID No: 2, or a functional variant or fragment thereof.

29. The method according to claim 17, wherein the cyclic polypeptide, derivative or analogue thereof comprises cyclic SEQ ID No: 3, or a functional variant or fragment thereof.

30. The method according to claim 29, wherein the derivative or analogue thereof has at least 70% sequence identity to SEQ ID No: 3.

31. The method according to claim 17, wherein the cyclic polypeptide, derivative or analogue thereof comprises cyclic SEQ ID No:4, or a functional variant or fragment thereof.

32. A Down's syndrome treatment pharmaceutical composition comprising a therapeutically effective amount of the cyclic polypeptide, derivative or analogue thereof according to claim 17, and a pharmaceutically acceptable vehicle.

33. A process for making the Down's syndrome treatment pharmaceutical composition according to claim 32, the process comprising combining a therapeutically effective amount of the cyclic polypeptide, derivative or analogue thereof according to claim 17, with a pharmaceutically acceptable vehicle.


SEQUENCE LISTING (4 sequences)

SEQ ID NO: 1 - protein, 614 aa, Homo sapiens
MRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATDTLDEAERQWKAEFHRWSSYMVHWKNQFDHYSKQDRCSDL

SEQ ID NO: 2 - protein, 30 aa, Homo sapiens
KAEFHRWSSYMVHWKNQFDHYSKQDRCSDL

SEQ ID NO: 3 - protein, 14 aa, Homo sapiens
AEFHRWSSYMVHWK

SEQ ID NO: 4 - protein, 15 aa, Homo sapiens
NQFDHYSKQDRCSDL